(19) United States
(12) Patent Application Publication
(10) Pub. No.: US 2026/0157777 A1
(43) Pub. Date: Jun. 11, 2026

(54) INTRAMEDULLARY NAIL WITH CONTINUOUS COMPRESSION

(71) Applicant: DePuy Synthes Products, Inc., Raynham, MA (US)

(72) Inventors: Ross Hamel, West Chester, PA (US); Rene Haag, Berwyn, PA (US)

(21) Appl. No.: 19/375,650

(22) Filed: Oct. 31, 2025

Related U.S. Application Data
(60) Provisional application No. 63/714,160, filed on Oct. 31, 2024.

(51) Int. Cl. A61B 17/72; A61B 17/88; A61B 17/56; A61B 17/68

(52) U.S. Cl. CPC A61B 17/7225; A61B 17/7233; A61B 17/8872; A61B 2017/564; A61B 2017/681

(57) ABSTRACT

An intramedullary nail can extend along a central axis. The intramedullary nail can include a first nail member, a bone anchor, and a biasing element. The first nail member can be configured to be positioned in a medullary canal of a bone. The first nail member can define a slot and at least one first bone fixation hole. The at least one bone fixation hole can be configured to receive a respective at least one first bone fixation element so as to couple the first nail member to a first bone portion of the bone. The bone anchor can be at least partially within the slot and configured to couple to a second bone portion of the bone. The biasing element can be configured to apply a biasing force to the bone anchor and the first nail member so as to urge at least one of the first nail member and the bone anchor toward the other of the first nail member and the bone anchor so as to create compression between the first and second bone portions.

### CROSS-REFERENCE TO RELATED APPLICATIONS

This claims the benefit of U.S. Patent Application Serial No. 63/714,160 filed October 31, 2024, the disclosure of which is hereby incorporated by reference as if set forth in its entirety herein.

### TECHNICAL FIELD

The present disclosure relates generally to bone intramedullary nails, and more specifically to intramedullary nails that couple bone segments to each other.

### BACKGROUND

Conventional intramedullary nails are configured to be inserted into the medullary canal of a bone that has been fractured so as to define a proximal bone segment and a distal bone segment that is separated from the proximal bone segment by a bone gap. Conventional intramedullary nails are elongate along a substantially central longitudinal axis, and include bone anchor holes that are configured to receive bone anchors. The bone anchor holes can include proximal bone anchor holes that extend through the proximal end of the intramedullary nail and distal bone anchor holes that extend through the distal end of the intramedullary nail. Thus, the intramedullary nail can be inserted into the medullary canal of the fractured long bone such that the proximal bone anchor holes are aligned with the proximal bone segment and the distal bone anchor holes are aligned with the distal bone segment on opposite sides of the bone gap. The bone segments can be positioned by a surgeon and the bone screws can be driven into the bone segments and the corresponding bone anchor holes so as to fasten the intramedullary nail to the fractured long bone and stabilize the proximal and distal bone segments relative to each other, thereby promoting healing. The relative positions of the bone segments are fixed once the bone screws are set using such traditional designs. Therefore, an improved intramedullary nail that compresses the bone segments against each other during the healing process is desired.

### SUMMARY

An intramedullary nail can extend along a central axis. The intramedullary nail can include a first nail member, a bone anchor, and a biasing element. The first nail member can be configured to be positioned in a medullary canal of a bone. The first nail member can define a slot and at least one first bone fixation hole. The at least one bone fixation hole can be configured to receive a respective at least one first bone fixation element so as to couple the first nail member to a first bone portion of the bone. The bone anchor can be at least partially within the slot and configured to couple to a second bone portion of the bone. The biasing element can be configured to apply a biasing force to the bone anchor and the first nail member so as to urge at least one of the first nail member and the bone anchor toward the other of the first nail member and the bone anchor, thereby creating compression between the first and second bone portions.

The biasing element can apply the biasing force to the nail body. The biasing element can apply the biasing force to the bone anchor. The slot and the fixation hole can be on opposite sides of a bone gap. In a further embodiment, the intramedullary nail includes an actuator coupled to the biasing element and the bone anchor such that the biasing element applies the biasing force to the actuator, thereby applying the biasing force to the bone anchor. The first nail member can include a channel configured to receive at least a portion of the actuator.

In a further embodiment, the intramedullary nail includes a pusher member configured to apply a second force to the bone anchor. The second force can be applied in a first direction and the biasing force can be applied in a second direction different from the first direction. The first direction can be opposite the second direction. The second force can be greater than the biasing force. The pusher member can be configured to move the second bone portion away from the first bone portion. The pusher member can be movable relative to the first nail member. The pusher member can be moveable in a longitudinal direction relative to the first nail member. The biasing element and the pusher member can be positioned on opposite sides of the bone anchor. The first nail member can include a bone fixation hole configured to receive a second bone anchor. The pusher member can include a pusher member slot configured to receive the second bone anchor. The biasing element can be a first biasing element and the intramedullary nail further can include a second biasing element. The first and second biasing elements can be positioned on opposite sides of the slot in a longitudinal direction. The second biasing element can be positioned between the slot and the first bone fixation hole in the longitudinal direction. The first bone portion and the second bone portion can be two pieces of a same bone. The first bone portion and the second bone portion can be different bones

A method of assembling an intramedullary nail can include coupling a pusher member to a nail body that has a first end and a second end opposite the first end along a central axis of the nail body, wherein the coupling step causes a majority of the pusher member to be disposed between a first slot of the first nail member and the first end. The method can include coupling an actuator to the first nail member such that at least a portion of the actuator extends from the first slot toward the second end. The method can include coupling a biasing element to each of the nail body and the biasing element, such that the biasing member urges the actuator to move substantially along the central axis toward the first end. Coupling the pusher member to the first nail member can include positioning the pusher member within a channel of the first nail member. The method can include coupling a connector to the first nail member such that the biasing element can be positioned between the actuator and the connector. The method can include moving the connector relative to the first nail member so as to increase potential energy of the biasing element.

A method of approximating a bone gap of a bone that separates the bone into a first bone portion and a second bone portion can include inserting an intramedullary nail into a medullary canal of the bone, inserting a bone anchor through a first slot in a nail body of the intramedullary nail into the first bone portion, such that the nail body can be movable relative to the bone anchor along a central axis of the nail body, inserting a first bone fixation element into a first bone fixation hole in the nail body and into the second bone portion so as to couple the first nail member to the second bone portion, and causing an actuator to bias against at least one of the bone anchor and the nail body the bone anchor so as to urge the at least one of the bone anchor and the nail body to move in a direction that decreases a distance from the first bone fixation hole to the bone anchor along the central axis. The first bone fixation hole can be sized substantially equal to the first bone fixation element. The first bone fixation hole can be elongate along the central axis.

### BRIEF DESCRIPTION OF THE DRAWINGS

The present application is further understood when read in conjunction with the appended drawings. For the purpose of illustrating the subject matter, there are shown in the drawings exemplary aspects of the subject matter; however, the presently disclosed subject matter is not limited to the specific methods, devices, and systems disclosed. In the drawings:

FIG. 1 is a front elevational view of an intramedullary nail and insertion instrument in accordance with one embodiment of the present disclosure;

FIG. 2 is a perspective view of the intramedullary nail of FIG. 1;

FIG. 3 is a sectional view of the intramedullary nail of FIG. 1;

FIG. 4 is a sectional view of the intramedullary nail and insertion instrument of FIG. 1;

FIG. 5 is a sectional view of the intramedullary nail and a portion of the insertion instrument of FIG. 1 in an initial first configuration;

FIG. 6 is a sectional view of a portion of the intramedullary nail of FIG. 1 in the initial configuration with a bone anchor;

FIG. 7 is a sectional view of a portion of the intramedullary nail of FIG. 1 in an intermediate configuration;

FIG. 8 is a sectional view of the intramedullary nail and a portion of the insertion instrument of FIG. 1 with the first and second bone portions in a reduced configuration;

FIG. 9 is a sectional view of the intramedullary nail and a portion of the insertion instrument of FIG. 1 with the first and second bone portions in a reduced configuration and first and second bone fixation elements coupled to the nail body;

FIG. 10 is a sectional view of the intramedullary nail and a portion of the insertion instrument of FIG. 1 with the pusher member in a second position relative to the nail body,

FIG. 11 is a sectional view of the intramedullary nail and a portion of the insertion instrument of FIG. 10 with first, second, and third bone fixation elements coupled to the nail body with the pusher member in a third retracted position relative to the nail body;

FIG. 12 is a perspective view of the pusher member of FIG. 4;

FIG. 13 is an exploded perspective view of the pusher member of FIG. 4;

FIG. 14 is a perspective view of the actuator of FIG. 4;

FIG. 15 is a perspective view of the first body of the insertion tool of FIG. 4;

FIG. 16A is a perspective view of the second body of the insertion tool of FIG. 4;

FIG. 16B is a sectional side elevation view of the second body of the insertion tool of FIG. 16A;

FIG. 17 is a perspective view of the third body of the insertion tool of FIG. 4; and

FIG. 18 is a flow chart illustrating a method of assembling an intramedullary nail.

Aspects of the disclosure will now be described in detail with reference to the drawings, wherein like reference numbers refer to like elements throughout, unless specified otherwise.

### DETAILED DESCRIPTION

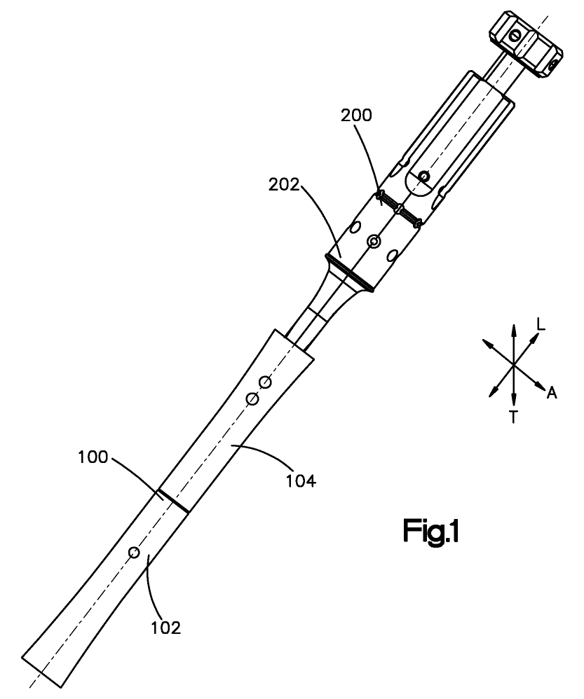

Referring to FIG. 1, an intramedullary nail 100 is shown. The intramedullary nail 100 can be configured to be positioned in a medullary canal. The intramedullary nail 100 can be coupled to a first bone portion 102 and a second bone portion 104. The intramedullary nail 100 can urge the first and second bone portions 102, 104 toward each other. The intramedullary nail 100 can move the first and second bone portions 102, 104 into contact with each other. The intramedullary nail 100 can provide a force that compresses the first and second bone portions 102, 104 against each other. The intramedullary nail 100 can provide a continuous compressive force to the first and second bone portions 102, 104 during the healing process. The portions of bone can be from a single fractured bone. The bone can be a long bone. The bone can be for example, a tibia, fibula, femur, metatarsals, and phalange, humerus, radius, ulna, or metacarpal. The portions of bone can be two different bones that are coupled together.

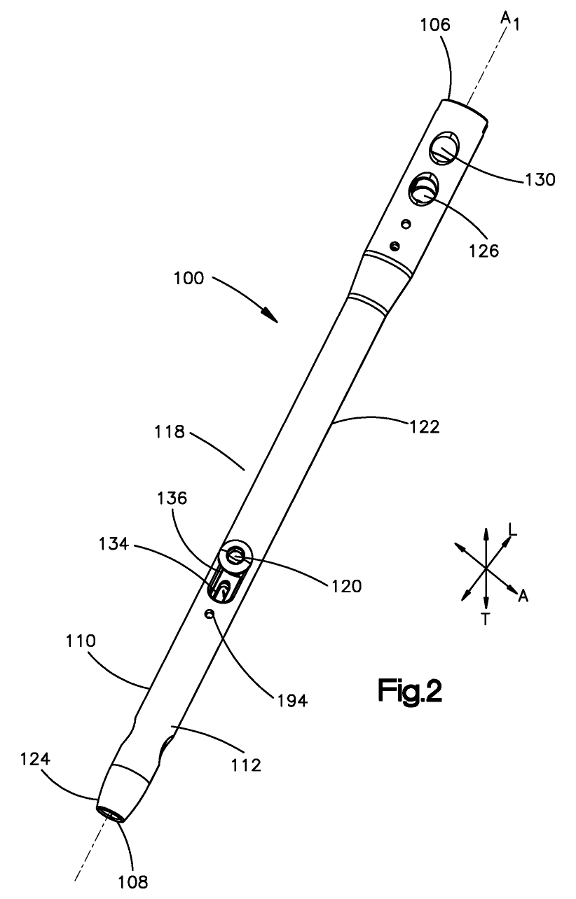

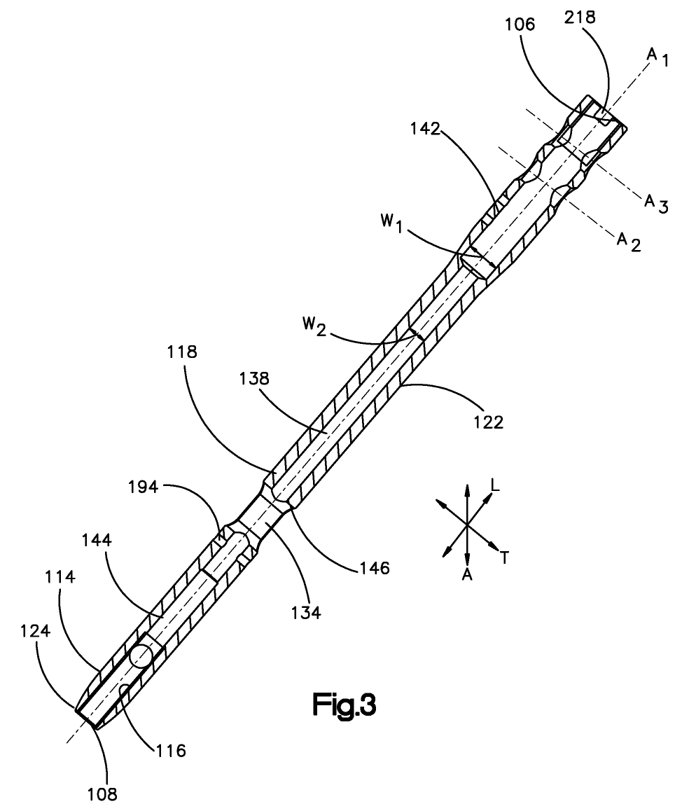

Referring to FIG. 2, the intramedullary nail 100 can be elongate along a central axis A1. The intramedullary nail 100 can include a first end 106 and a second end 108 spaced from the second end in a longitudinal direction L. The second end 108 can be spaced from the first end 106 in a distal direction. The first end 106 can be opposite the second end 108 along the central axis A1. The intramedullary nail 100 can include a first side 110 and a second side 112 spaced from the first side 110 in a lateral or radial direction A. The intramedullary nail 100 can include a first or outer surface 114 and a second or inner surface 116 spaced from the first surface 114 in a transverse or radial direction T (FIG. 3). The lateral direction A can be perpendicular to the longitudinal direction L. The transverse direction T can be perpendicular to each of the longitudinal direction L and the lateral direction A. The central axis A1 can extend in the longitudinal direction L.

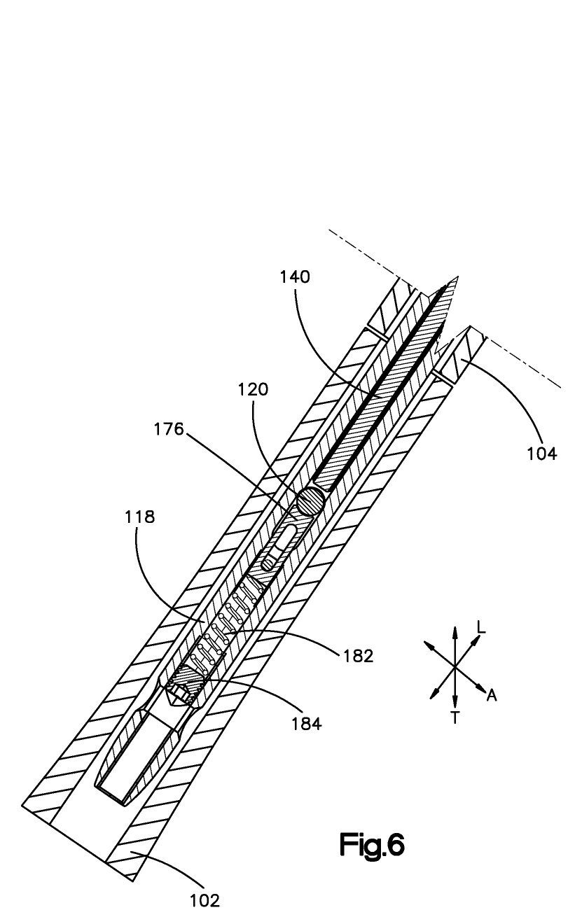

Referring to FIG. 6, the intramedullary nail 100 can include a nail body 118 fixed to the first bone portion 102. The intramedullary nail 100 can include a bone anchor 120 fixed to the first bone portion 102. The bone anchor 120 can be movable relative to the nail body 118. The bone anchor 120 can be a threaded fastener such as a screw, or can be a rivet, nail, adhesive, or any suitable alternative constructed bone fixation element as desired. An actuator 176 can be configured to move the bone anchor 120 relative to the nail body 118. The first and second bone portions 102, 104 can move relative to each other as the bone anchor 120 and the nail body 118 move relative to each other. The nail body 118 and bone anchor 120 can be movable relative to each other in the longitudinal direction L. At least one of the nail body 118 and the bone anchor 120 can be movable relative to the other of the nail body 118 and the bone anchor 120 along the central axis A1.

Referring to FIG. 2, the nail body 118 can include an outer wall 122. The outer wall 122 can have a cylindrical cross-sectional shape taken along a plane transverse to the longitudinal direction L. The plane can extend in the transverse direction T and the lateral direction A. The outer wall 122 can be sized and shaped such that the nail body 118 can be inserted into a medullary canal. The outer wall 122 can have a cylindrical shape. The nail body 118 can include an end portion 124 (FIG. 3) configured to facilitate insertion of the nail body 118 into the medullary canal. The end portion 124 can be tapered inwardly in the longitudinal direction L to facilitate insertion. The end portion 124 can be tapered inwardly in a direction from the first end 106 toward the second end 108. The nail body 118 can be one or more pieces.

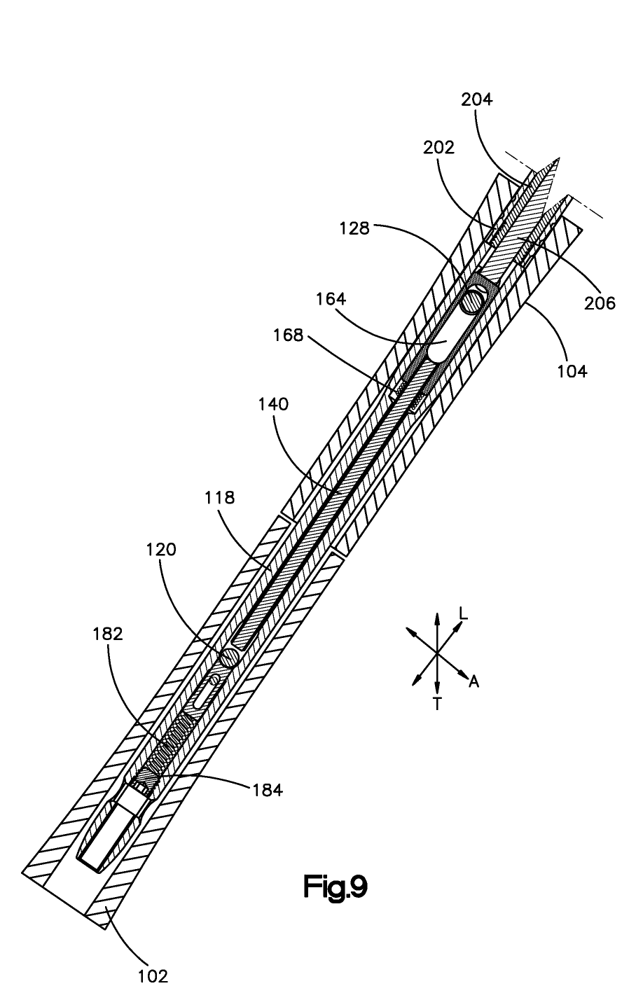

The nail body 118 can be configured to be fixed to the first bone portion 102. Referring to FIG. 2, the nail body 118 can include a first bone fixation hole 126 configured to receive a first bone fixation element 128 (FIG. 9) so as to fix the nail body 118 to the second bone portion 104. The first bone fixation hole 126 can extend through the outer wall 122. The first bone fixation hole 126 can extend through the outer wall 122 along a first bone fixation hole central axis A2 (FIG. 3). The central axis A2 can be transverse to the central axis A1. The central axis A2 can be perpendicular to the central axis A1. The central axis A2 can be elongate along the transverse direction T. The first bone fixation hole 126 can extend from the first surface 114 of the outer wall 122 toward the second surface 116. The first bone fixation hole 126 can extend from the first surface 114 to the second surface 116. The first bone fixation hole 126 can extend through each of the first and second surfaces 114, 116.

The first bone fixation element 128 can be a threaded fastener such as a screw, or can be a rivet, nail, adhesive, or any suitable alternative constructed bone fixation element as desired. The first bone fixation element 128 can fix the position of the nail body 118 in the longitudinal direction L relative to the second bone portion 104. The first bone fixation element 128 can threadedly engage the first bone fixation hole 126. The first bone fixation element 128 can include a head that engages a sidewall of the first bone fixation hole 126 thereby locking the first bone fixation element 128 in the first bone fixation hole 126. The first bone fixation hole 126 can be sized and shaped to receive the first bone fixation element 128 such that the position of the nail body 118 is fixed in the longitudinal direction L. The first bone fixation hole 126 can be sized such that the first bone fixation element 128 fixes the position of the nail body 118 in the longitudinal direction L when the first bone fixation element 128 is in the first bone fixation hole 126. In some examples, the first bone fixation element 128 has a diameter sized such that the first bone fixation element 128 is configured to be inserted in the first bone fixation hole 126. The first bone fixation element 128 has a length greater than that of the bone fixation hole 126, and thus also greater than the diameter of the nail body, so that the first bone fixation element 128 can fully engage the bone. In some examples, the length of the first bone fixation element 128 can be greater than the dimension of the bone along a direction that is defined by the length of the first bone fixation element 128. As used herein, “about” can mean within 1%, 2%, 3%, 4%, 5%, or 10% of the stated value.

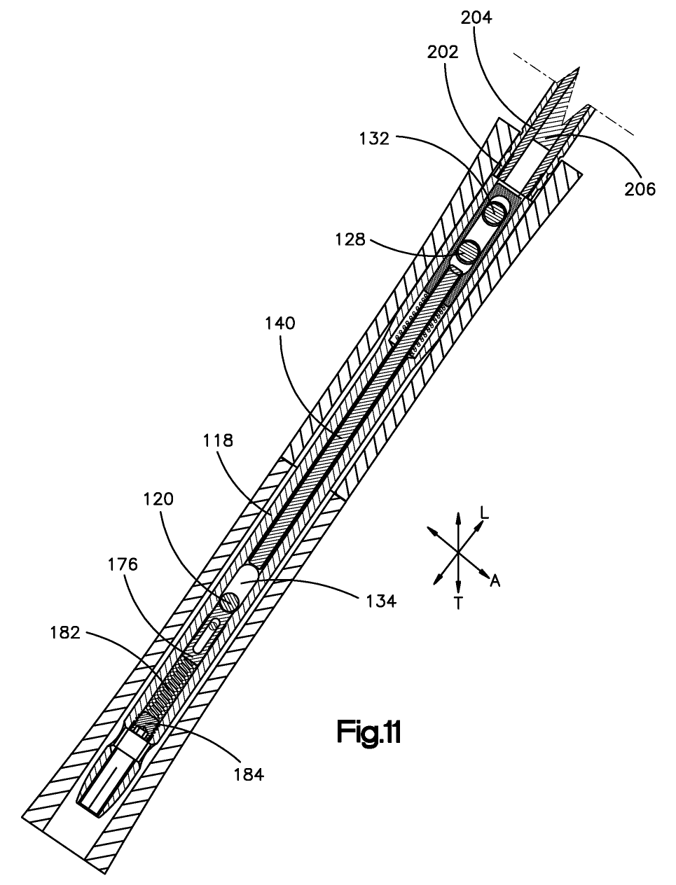

The nail body 118 can include a second bone fixation hole 130 configured to receive a second bone fixation element 132 (FIG. 11) so as to further fix the nail body 118 to the second bone portion 104. The second bone fixation hole 130 can extend through the outer wall 122. The second bone fixation hole 130 can extend through the outer wall 122 along a second bone fixation hole central axis A3 (FIG. 3). The central axis A3 can be transverse to the central axis A1. The central axis A3 can be perpendicular to the central axis A1. The central axis A3 can be elongate along the transverse direction T. In some examples, the central axis A3 is parallel to the central axis A2 in the transverse direction T. In some examples, the central axis A3 is transverse to the central axis A2 in the transverse direction T. In some examples, the central axis A3 is parallel to the central axis A2 in the lateral direction A. In some examples, the central axis A3 is transverse to the central axis A2 in the lateral direction A. Each of the central axis A2 and the central axis A3 can be oblique and non-perpendicular to the central axis A1, or can alternatively be perpendicular to the central axis A1. The second bone fixation hole 130 can be positioned between the first bone fixation hole 126 and the first end 106 in the longitudinal direction L.

The second bone fixation hole 130 can extend from the first surface 114 of the outer wall 122 toward the second surface 116. The second bone fixation hole 130 can extend from the first surface 114 to the second surface 116. The second bone fixation hole 130 can extend through each of the first and second surfaces 114, 116.

The second bone fixation element 132 can be a threaded fastener such as a screw, or can be a rivet, nail, adhesive, or any suitable alternative constructed bone fixation element as desired. The second bone fixation element 132 can fix the position of the nail body 118 in the longitudinal direction L relative to the second bone portion 104. The second bone fixation element 132 can threadedly engage the second bone fixation hole 130. The second bone fixation element 132 can include a head that engages a sidewall of the second bone fixation hole 130 thereby locking the second bone fixation element 132 in the second bone fixation hole 130. The second bone fixation hole 130 can be sized and shaped to receive the second bone fixation element 132 such that the position of the nail body 118 is fixed in the longitudinal direction L. The second bone fixation hole 130 can be sized such that the second bone fixation element 132 fixes the position of the nail body 118 in the longitudinal direction L when the second bone fixation element 132 is in the second bone fixation hole 130. In some examples, the second bone fixation element 132 has a diameter sized such that the second bone fixation element 132 is configured to be inserted in the second bone fixation hole 130. The second bone fixation element 132 has a length greater than that of the second bone fixation hole 130, and thus also greater than the diameter of the nail body, so that the second bone fixation element 132 can fully engage the bone. In some examples, the length of the second bone fixation element 132 can be greater than the dimension of the bone along a direction that is defined by the length of the second bone fixation element 132.

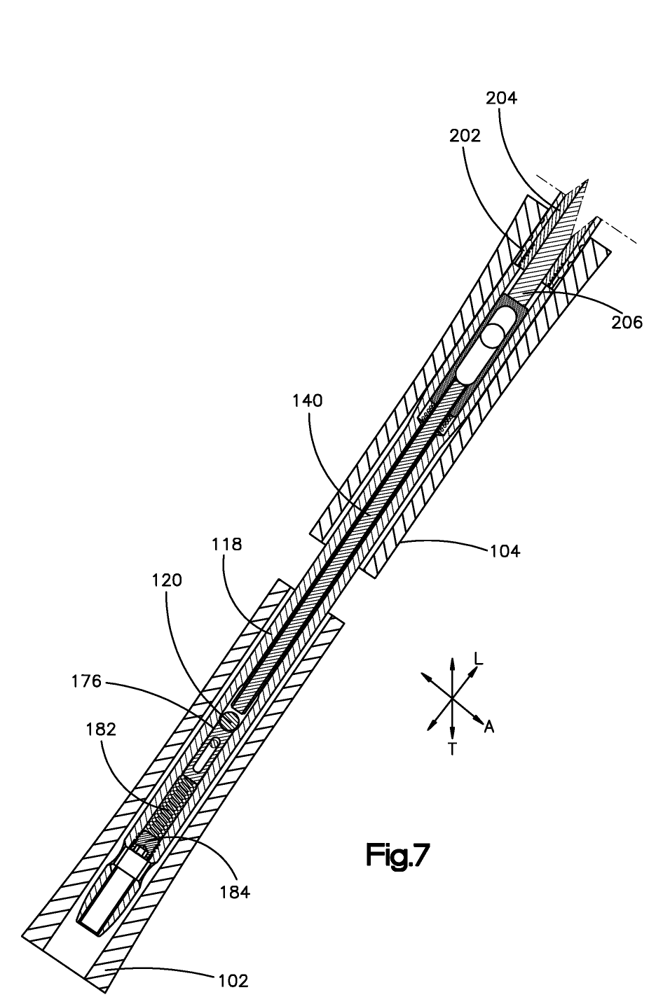

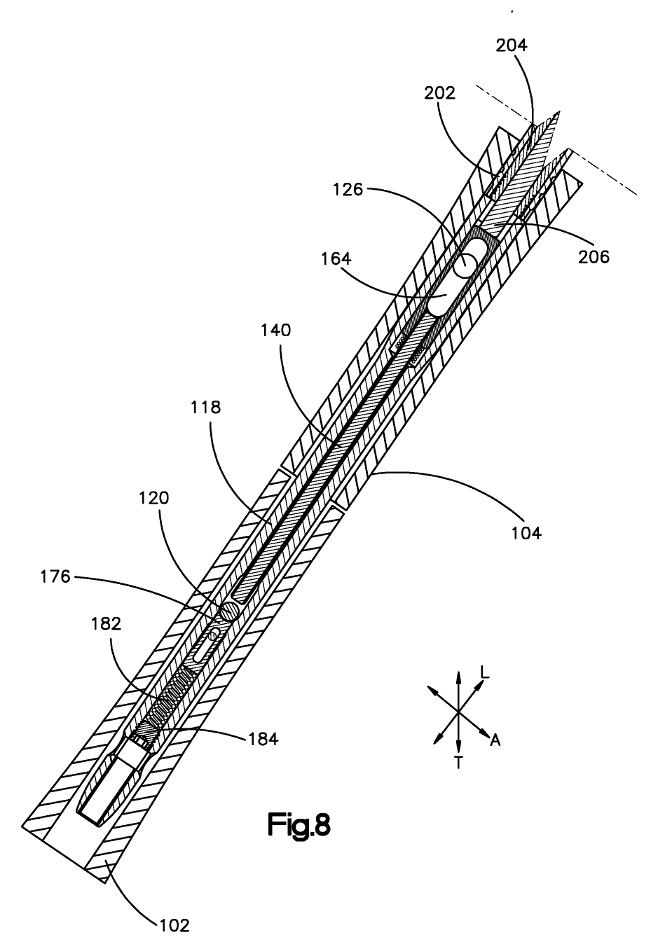

The nail body 118 can be configured to receive the bone anchor 120. The bone anchor 120 can be a threaded fastener, nail, rivet, or adhesive. The nail body 118 can include a first slot 134 (FIG. 2) configured to receive the bone anchor 120. A first slot sidewall 136 can define the first slot 134. The first slot 134 can extend through the outer wall 122 of the nail body 118. The first slot 134 can be elongate in the longitudinal direction L such that the nail body 118 is moveable in the longitudinal direction L relative to the bone anchor 120 when the bone anchor 120 is within the first slot 134. At least one of the bone anchor 120 and the nail body 118 can be movable relative to the other of the bone anchor 120 and the nail body 118 such that relative position of the bone anchor 120 and the first slot 134 changes from a first position (FIG. 7) to a second position (FIG. 8). At least one of the first bone portion 102 and the nail body 118 can move relative to the other of the first bone portion 102 and the nail body 118 as the bone anchor 120 moves relative to the first slot 134. The bone anchor 120 can contact a distal portion of the first slot sidewall 136 in the first position. The distal portion can be closer to the second end 108 than the first end 106. The bone anchor 120 can contact a proximal portion of the first slot sidewall 136 in the second position. The proximal portion can be closer to the first end 106 than the second end 108. Alternatively, the bone anchor 120 can be spaced from the second end of the first slot sidewall 136 in the second position. The bone anchor 120 can be spaced from the first end of the first slot sidewall 136 in the first position.

The nail body 118 can be configured to receive an actuation assembly. Referring to FIG. 3, the nail body 118 can include a channel 138. The outer wall 122 of the nail body 118 can define the channel 138. The channel 138 can be elongate in the longitudinal direction. The channel 138 can be elongate along the central axis A1. The channel 138 can extend from the first end 106 toward the second end 108. The channel 138 can extend from the first end 106 to the second end 108. The channel 138 can extend through each of the first end 106 and the second end 108.

The channel 138 can include a first channel portion 142 and a second channel portion 144. The first channel portion 142 can have a width W1. The width W1 can be measured in a plane including the transverse direction T and the lateral direction A. The second channel portion 144 can have a width W2. The width W1 can be equal to the width W2. The width W1 can be greater than the width W2. The width W1 can be less than the width W2. The first channel portion 142 can have a first channel length in the longitudinal direction L. The second channel portion 144 can have a second channel length in the longitudinal direction L. The second channel length can be greater than the first channel length. The second channel length can be a majority of the length of the nail body 118. A shoulder 146 can separate the first channel portion 142 from the second channel portion 144. The shoulder 146 and the outer wall 122 can be a monolithic element. The width of the outer wall 122 can be greater at the second channel portion 144 than the width of the outer wall 122 at the first channel portion 142.

The actuation assembly can include an actuator 176 and a biasing element 182. Referring to FIG. 6, the actuator 176 can be configured to move at least one of the bone anchor 120 and the nail body 118 relative to the other of the bone anchor 120 and the nail body 118. The actuator 176 can be a traveler. The actuator 176 can be disposed in the channel 138. The biasing element 182 can urge the actuator 176 into contact with the bone anchor 120. The biasing element 182 can include a first end coupled to the actuator 176. The biasing element 182 can include a second end coupled to a connector 184. The biasing element 182 can be compressed between the actuator 176 and the connector 184. The connector 184 can be fixed to the nail body 118 such that the biasing element 182 urges the bone anchor 120 so as to increase a distance between the bone anchor 120 and the connector 184. The biasing element 182 can urge the bone anchor 120 so as to decrease the distance between the bone anchor 120 and the first bone fixation hole 126. The distance between the bone anchor 120 and the first bone fixation hole 126 can be measured along the central axis A1. The distance between the bone anchor 120 and the first bone fixation hole 126 can be measured along the longitudinal direction L.

The connector 184 can be at least temporarily fixed relative to the nail body 118 in the longitudinal direction. In some examples, the connector 184 is fixed to the nail body 118. In other examples, the connector 184 is movably coupled to the nail body 118 so as to adjust a position of the connector 184. The connector 184 can be fixed relative to the nail body 118 when the biasing element 182 urges the bone anchor 120 so as to increase a distance between the bone anchor 120 and the connector 184. The connector 184 can be movable in the longitudinal direction L relative to the nail body 118. The connector 184 can be rotatable about the central axis A1 relative to the nail body 118. The connector 184 can be threadedly engaged with the nail body 118. The connector 184 can be a set screw. The connector 184 can be positioned in the channel 138. The connector 184 can be adjustable so as to adjust a force provided by the biasing element 182. For example, movement of the connector 184 toward the first end 106 can compress the biasing element 182 thereby increasing the potential energy of the biasing element 182. The connector 184 can be positioned in the second channel portion 144. The connector 184 and the biasing element 182 can each be positioned in the second channel portion 144. The connector 184 can be positioned between the second end 108 and the biasing element 182.

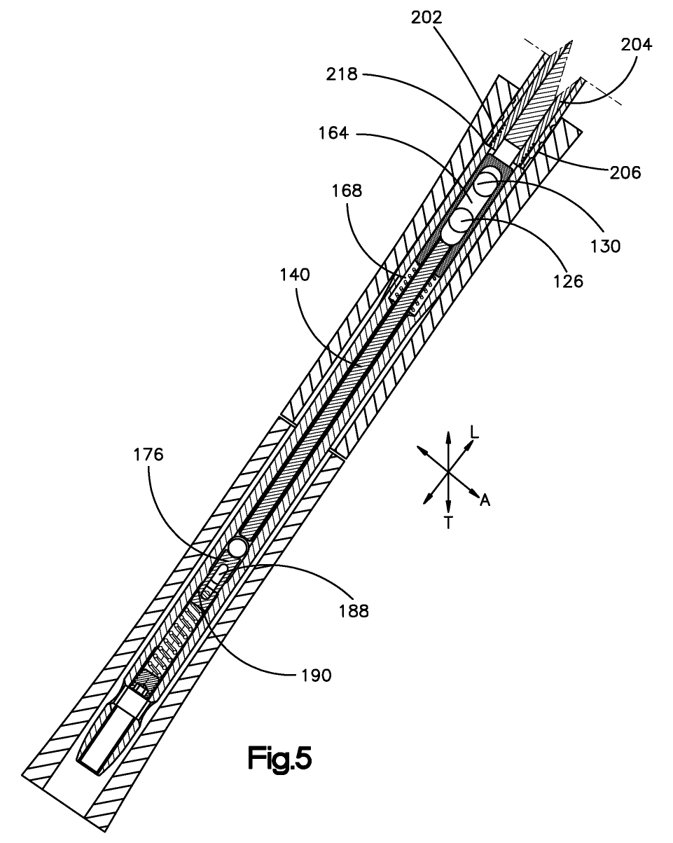

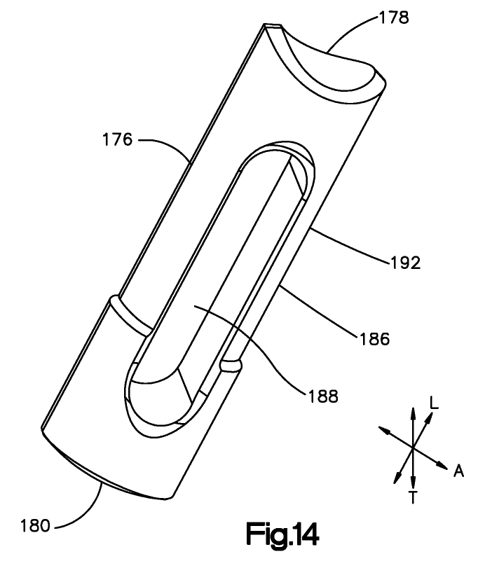

Referring to FIG. 14, the actuator 176 can include a first end 178 configured to engage the bone anchor 120. The first end 178 can define a radius configured to engage the bone anchor 120. The actuator 176 can include a second end 180 opposite the first end in the longitudinal direction L. The biasing element 182 can engage the second end 180 of the actuator 176. The actuator 176 can include an actuator body 186. The actuator body 186 can be elongate between the first and second ends 178, 180. An actuator slot 188 can extend through the body 186. The actuator slot 188 can extend through the body 186 in the transverse direction T. Alternatively, the actuator slot 188 can be a track that does not extend through the body 186. The actuator slot 188 can be configured to receive an alignment member 190 (FIG. 5). The alignment member 190 can limit relative movement between the actuator 176 and the nail body 118. Referring to FIG. 5, the first end 178 of the actuator 176 can be distal to the proximal end of the first slot 134 when the alignment member 190 engages the second end of the actuator slot 188. The alignment member 190 can bottom out within the actuator slot 188 to leave space through which the bone anchor 120 can be inserted into the first slot 134. The alignment member 190 can be a pin, fastener, or dowel. The alignment member 190 can be a protrusion of the nail body 118. The alignment member 190 can be fixed to the nail body 118. The alignment member 190 can be fixed to the nail body 118 in the longitudinal direction L.

In some examples, the nail body 118 includes an opening 194 (FIGS. 2 and 3) configured to receive the alignment member 190. In other examples, the alignment member 190 and the nail body 118 are a monolithic construct. The opening 194 can be positioned between the second end 108 and the first slot 134.

The actuator slot 188 can be defined by an actuator slot sidewall 192. The actuator slot 188 can be elongate in the longitudinal direction L such that the actuator 176 is moveable in the longitudinal direction L relative to the alignment member 190 when the alignment member 190 is within the actuator slot 188. At least one of the alignment member 190 and the actuator 176 can be movable relative to the other of the alignment member 190 and the actuator 176 such that relative position of the alignment member 190 and the actuator slot 188 changes from a first position (FIG. 7) to a second position (FIG. 8). The alignment member 190 can contact a proximal portion of the actuator slot sidewall 192 in the first position. The proximal portion can be closer to the first end 106 than the second end 108. The alignment member 190 can contact a distal portion of the actuator slot sidewall 192 in the second position. The distal portion can be closer to the second end 108 than the first end 106. Alternatively, the alignment member 190 can be spaced from the actuator slot sidewall 192 in the second position. The alignment member 190 can be spaced from the actuator slot sidewall 192 in the first position.

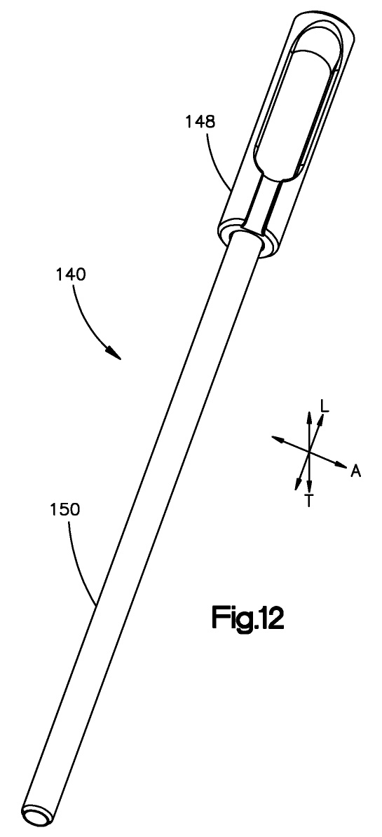

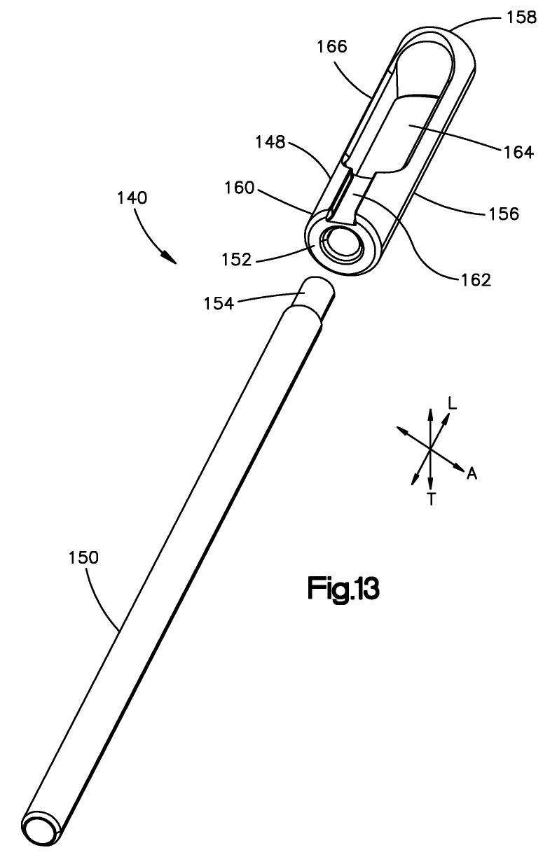

Referring to FIGS. 12 and 13, the intramedullary nail 100 can include a pusher member 140. The pusher member 140 can be configured to move the bone anchor 120 relative to the nail body 118. The pusher member 140 can move the bone anchor 120 and the first bone portion 102 relative to the nail body 118. The pusher member 140 can be coupled to the nail body 118. At least one of the first and pusher members 118, 140 can be movable relative to the other of the first and pusher members 118, 140. At least one of the first and pusher members 118, 140 can be movable relative to the other of the first and pusher members 118, 140 in the longitudinal direction L. In some examples, the first and pusher members 118, 140 are rotationally fixed relative to each other. In other examples, at least one of the first and pusher members 118, 140 can rotate relative to the other of the first and pusher members 118, 140. The pusher member 140 can be positioned in the channel 138. The pusher member 140 can be movable within the channel 138. The pusher member 140 and the actuator 176 can be disposed on opposing sides of the bone anchor 120. The pusher member 140 can be configured to move the bone anchor 120 relative to the nail body 118 in a first direction. The actuator 176 can be configured to move the bone anchor 120 relative to the nail body 118 in a second direction. The second direction can be opposite the first direction. The first direction can be the distal direction. The second direction can be the proximal direction.

The pusher member 140 can include a shaft 150 and a receiver 148. The receiver 148 can be positioned in the first channel portion 142. The shaft 150 can be positioned in the second channel portion 144. In some examples, the shaft 150 is detachably coupled to the receiver 148. In other examples, the shaft 150 is fixed to the receiver 148. The shaft 150 can be configured to engage the bone anchor 120.

Referring to FIG. 13, the receiver 148 can include a receiver engagement feature 152. The receiver engagement feature 152 can be configured to engage a shaft engagement feature 154. In some examples, the shaft engagement feature 154 threadedly engages the receiver engagement feature 152. In other examples, the shaft engagement feature 154 can be fixed to the receiver engagement feature 152 via press fit, adhesive, weld, or fastener.

The receiver 148 can include a body 156 with a first end 158 and a second end 160. The first end 158 can be opposite the second end 160 in the longitudinal direction L. The receiver engagement feature 152 can be a recess that extends from the second end 160 toward the first end 158. The receiver 148 can include an alignment feature 162. The alignment feature 162 can engage a corresponding feature on the nail body 118. The alignment feature 162 can maintain an alignment of the first and pusher members 118, 140 relative to each other. The alignment feature 162 can prevent relative rotation between the first and pusher members 118, 140. The alignment feature 162 can be a recess in an outer surface of the body 156. The alignment feature 162 can be configured to receive a protrusion of the nail body 118. Alternatively, the alignment feature 162 can be a protrusion configured to be received by a recess of the nail body 118.

A second slot 164 can extend through the body 156 of the receiver. The second slot 164 can be configured to receive at least one of the first and second bone fixation elements 128, 132. The second slot 164 can be configured to receive each of the first and second bone fixation elements 128, 132. The second slot 164 can be defined by a second slot sidewall 166. The second slot 164 can be elongate in the longitudinal direction L such that the receiver 148 is moveable in the longitudinal direction L relative to at least one of the first and second bone fixation elements 128, 132 when at least one of the first and second bone fixation elements 128, 132 are within the second slot 164. At least one of the receiver 148 and the first bone fixation element 128 can be movable relative to the other of the receiver 148 and the first bone fixation element 128 such that position of the first bone fixation element 128 within the second slot 164 changes from a first position (FIG. 7) to a second position (FIG. 8). The first bone fixation element 128 can contact a distal portion of the second slot sidewall 166 in the first position. Alternatively, the first bone fixation element 128 can be spaced from the distal portion of the second slot sidewall 166 in the first position. The distal portion can be closer to the second end 108 than the first end 106. The first bone fixation element 128 can contact a proximal portion of the second slot sidewall 166 in the second position. The proximal portion can be closer to the first end 106 than the second end 108. Alternatively, the first bone fixation element 128 can be spaced from the proximal end of the second slot sidewall 166 in the second position. The pusher member 140 can be moveable relative to each of the first and second bone portions 102, 104 when the bone anchor 120 is coupled to the first bone portion 102 and the first bone fixation element 128 is coupled to the second bone portion 104.

The intramedullary nail 100 can include a second biasing element 168 (FIG. 5) configured to urge the pusher member 140 relative to the nail body 118. The second biasing element 168 can urge the pusher member 140 toward the first end 106 of the nail body 118. The second biasing element 168 can urge the pusher member 140 toward the first position (FIG. 7). The second biasing element 168 can be positioned between the receiver 148 and the shoulder 146. The receiver 148 and the shoulder 146 can be disposed on opposite sides of the second biasing element 168 in the longitudinal direction L. The receiver 148 can be spaced form the shoulder 146 when the pusher member 140 is in at least one of the first and second positions. The second biasing element 168 can be positioned in the first channel portion 142. In some examples, the intramedullary nail 100 can be devoid of the second biasing element 168.

When the intramedullary nail 100 includes the second biasing element 168, the second biasing element 168 can be a resilient element (e.g., rubber). The second biasing element 168 can be made from nickel-titanium (Nitinol) such that the second biasing element 168 expands in response to temperature change (e.g., as the biasing element increases temperature). The second biasing element 168 can be compressed gas. The second biasing element 168 can be compressible. The second biasing element 168 can be compressed between the nail body 118 and the pusher member 140. The second biasing element 168 can be positioned in the first channel portion 142. The second biasing element 168 can have a cylindrical cross-sectional shape. The second biasing element 168 can define an internal opening such that the shaft 150 extends through the second biasing element 168.

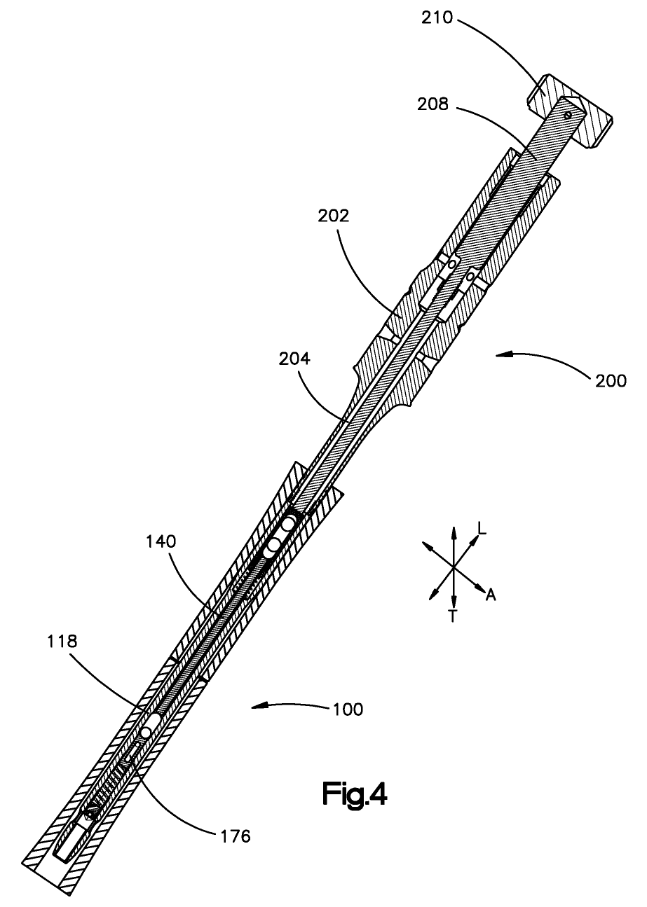

An insertion tool 200 can be configured to move at least one of the first and pusher members 118, 140 relative to the other of the first and pusher members 118, 140. Referring to FIG. 4, the insertion tool 200 can include a first body 202 and a second body 204. The insertion tool 200 can include a third body 208. The first body 202 can be rotationally fixed relative to the nail body 118 . The second body 204 can be coupled to the nail body 118 such that the second body 204 and nail body 118 are fixed to each other in the longitudinal direction L. The third body 208 can be movable relative to the nail body 118 so as to move the pusher member 140 in the longitudinal direction L.

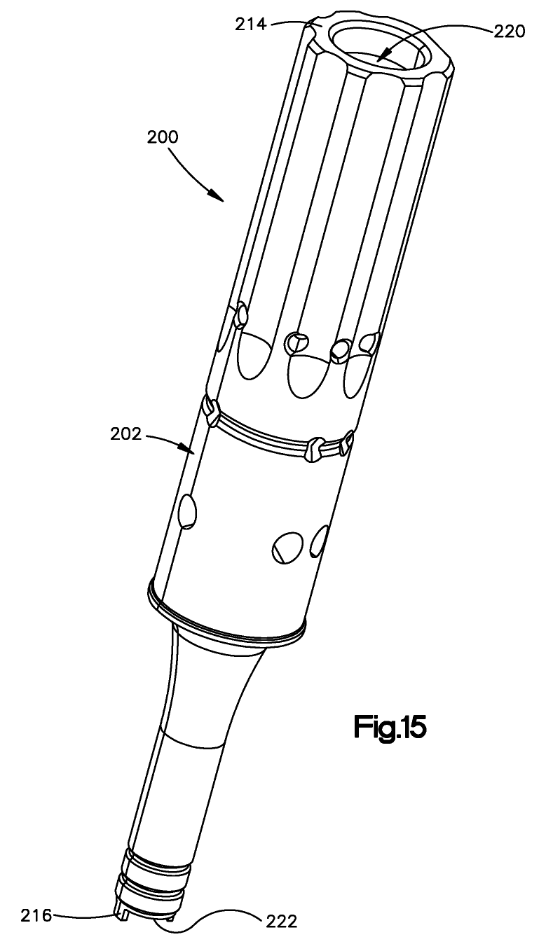

The first body 202 can be an outer body. The first body 202 can be graspable by a user. At least a portion of the first body 202 can be configured to engage the nail body 118. Referring to FIG. 15, the first body 202 can include a first end 212 and a second end 214. The second end 214 can be opposite the first end 212 along a central axis. The central axis can extend in the longitudinal direction L. The second end 214 can be spaced from the first end 212 in a distal direction. The first end 212 can abut the first end 106 of the nail body 118. The first end 212 can include a first body engagement feature 216 configured to engage a corresponding engagement feature 218 (FIG. 5) on the nail body 118. At least one of the first body engagement feature 216 and the engagement feature 218 can be a protrusion and the other of the first body engagement feature 216 and the engagement feature 218 can be a recess configured to receive the protrusion. In other examples, the first body engagement feature 216 and the engagement feature 218 can be magnets. The first body engagement feature 216 and the engagement feature 218 can rotationally fix the first body 202 relative to the nail body 118. The first body 202 can include a channel 220 that extends from the first end 212 to the second end 214.

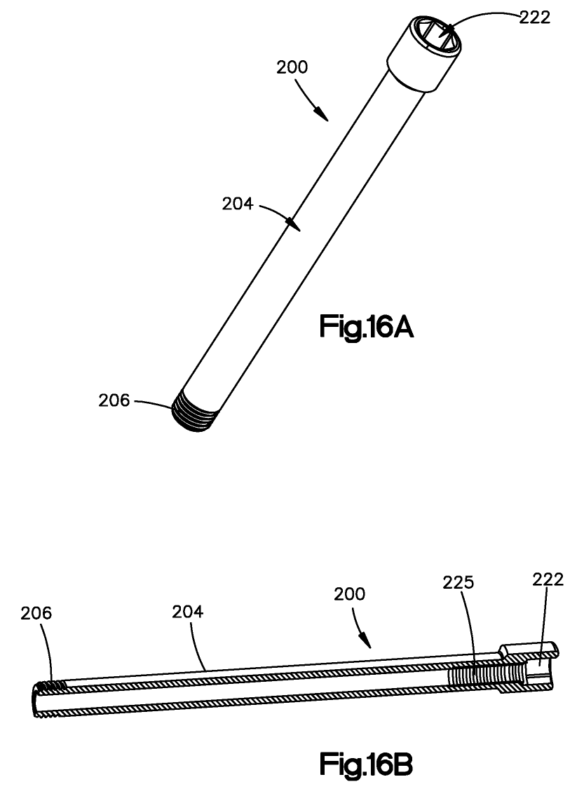

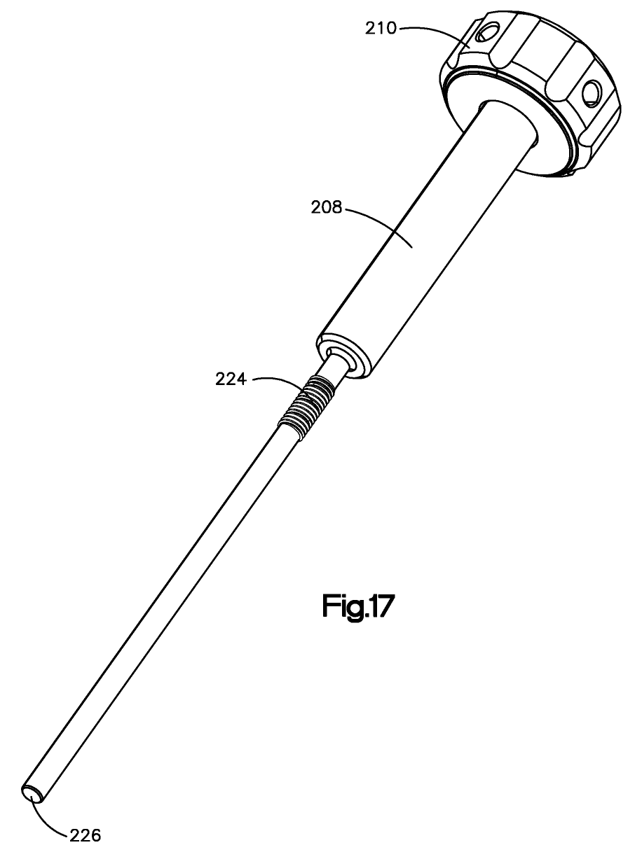

Referring to FIG. 4, at least a portion of the second body 204 can be positioned within the channel 220. The second body 204 can be movable within the channel 220. The second body 204 can be movable in the longitudinal direction L within the channel 220. The second body 204 can be rotatable within the channel 220. The second body 204 can be rotatable about the central axis when the second body 204 is within the channel 220. The second body 204 can be rotatable about the central axis A1 relative to the first body 202. Referring to FIGS. 16A-16B, the second body 204 can include a second body engagement feature 206. The second body engagement feature 206 can be configured to engage a corresponding engagement feature on the nail body 118. The second body engagement feature 206 can threadedly engage the nail body 118. The nail body 118 can be fixed in the longitudinal direction L relative to the first and second bodies 202, 204 when the second body engagement feature 206 is coupled to the nail body 118. A tool (not shown) can be inserted into the channel 220 through the second end 214 of the first body 202 and into a drive recess 222 of the second body 204 to move the second body engagement feature 206 into engagement with the nail body 118. The tool can thread the second body 204 into the intramedullary nail 100. The second body 204 can also have an internal threaded portion 225 that threadedly engages a third body engagement feature 224, which can define threads, of the third body 208, which described in more detail below with reference to FIGS. 4 and 17. At least a portion of the second body 204 can extend from the second end 214 of the first body 202 in the distal direction when the second body engagement feature 206 is coupled to the nail body 118. The first and second bodies 202, 204 can be fixed relative to the nail body 118 when the second body engagement feature 206 is coupled to the nail body 118. The second body 204 can define a second body channel that defines the drive recess 222.

Referring to FIG. 4, the third body 208 can be disposed within the second body channel 222. The second body 204 can be positioned between the first and third bodies 202, 208 in the lateral direction A. The third body 208 can be removably coupled to the second body 204. The third body 208 can be movable in the longitudinal direction L relative to the second body 204. The third body 208 can be rotatable relative to the second body 204. The third body 208 can be rotatable relative to the first body 202. The third body 208 can be movable in the longitudinal direction L relative to each of the first and second bodies 202, 204. An actuation feature 210 can be coupled to the third body 208. The actuation feature 210 can be engageable by a user to move the third body 208. A user can grasp the first body 202 with one hand and grasp the actuation feature 210 with a second hand to move the third body 208 relative to the first body 202. The third body 208 can be rotationally fixed to the actuation feature 210. The third body 208 can include a third body engagement feature 224 (see FIG. 17). The third body engagement feature 224 can be configured to engage at least one of the first and second bodies 202, 204. The third body engagement feature 224 can be a thread to threadedly engage at least one of the first and second bodies 202, 204 such that the third body 208 moves in the longitudinal direction L relative to the first and second bodies 202, 204 in response to rotation of the third body 208. The third body 208 can engage the pusher member 140 as the third body 208 moves in the longitudinal direction L relative to the second body 204. An end 226 of the third body 208 can engage the pusher member 140 as the third body 208 moves in the longitudinal direction L relative to the second body 204.

A method of coupling the first and second bone portions 102, 104 to each other can include coupling the insertion tool 200 to the intramedullary nail 100. Coupling the insertion tool 200 to the intramedullary nail 100 can include engaging the nail body 118 with the second body engagement feature 206. The intramedullary nail 100 can then be positioned in the medullary canal of the first and second bone portions 102, 104. In some examples, the first and second bone portions 102, 104 are reduced when the intramedullary nail 100 is inserted into the medullary canal (FIG. 5). The bone anchor 120 can be inserted into the first slot 134 when the intramedullary nail 100 is within the medullary canal.

The third body 208 can be moved relative to the second body 204 after the bone anchor 120 is within the first slot 134. The third body 208 can move the pusher member 140 relative to the nail body 118 as the third body 208 moves in the distal direction. The third body 208 can move the pusher member 140 into engagement with the bone anchor 120 as the third member 208 moves in the distal direction. At least one of the first and second bone portions 102, 104 can move away from the other of the first and second bone portions 102, 104 as the pusher member 140 moves in the distal direction relative to the nail body 118 (FIG. 6). At least one of the intramedullary nail 100 and the second bone portion 104 can then be moved relative to the other of the intramedullary nail 100 and the second bone portion 104 such that the first and second bone portions 102, 104 are reduced (FIG. 8). The bone anchor 120 can be in a distal portion of the first slot 134 when the first and second bones are reduced.

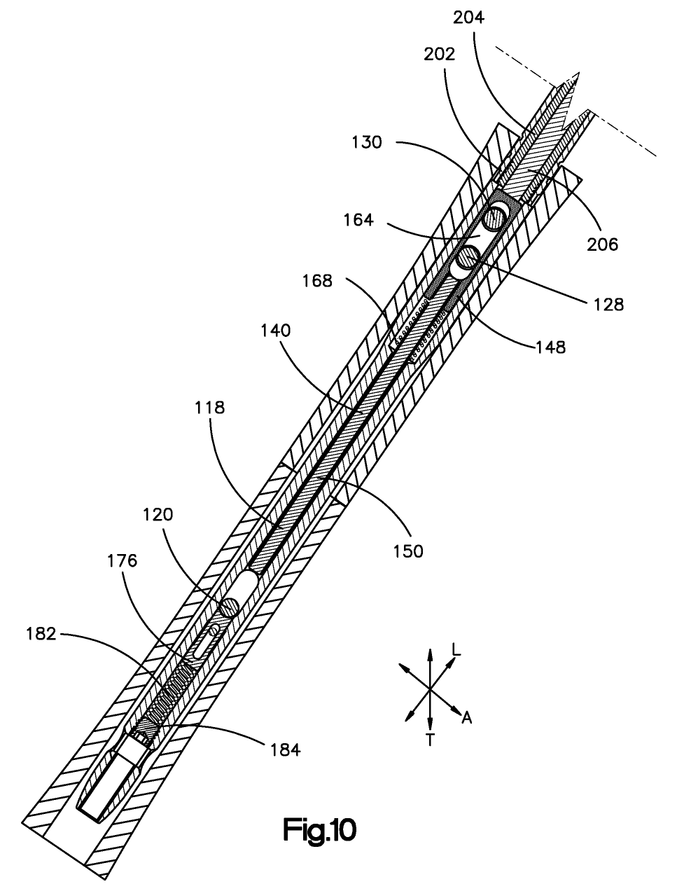

The first bone fixation element 128 can be positioned in the first bone fixation hole 126 when the first and second bone portions 102 are reduced (FIG. 9). The pusher member 140 can occlude the second bone fixation hole 130 when the third body 208 is in a distal position. The third body 208 of the insertion tool 200 can then be moved in the proximal direction. The second biasing element 168 can move the pusher member 140 in the proximal direction relative to the nail body 118 when the third body 208 moves in the proximal direction. The second biasing element 168 can move the pusher member 140 relative to the nail body 118 such that the second slot 164 is aligned in the longitudinal direction with the second bone fixation hole 130 (FIG. 10). The second bone fixation element 132 can then be inserted into the second bone fixation hole 130 (FIG. 11). The third body 208 can then be moved proximally such that the third body 208 disengages from the pusher member 140. The second body 204 can then be disengaged from the nail body 118 and the insertion tool 200 disengaged from the intramedullary nail 100. The bone anchor 120 can be spaced from the proximal end of the first slot 134 when the first and second bone portions 102, 104 are reduced such that the actuator 176 urges the first and second bone portions toward each other such that the first and second bone portions 102, 104 provide a continuous compressive force against each other during the healing process.

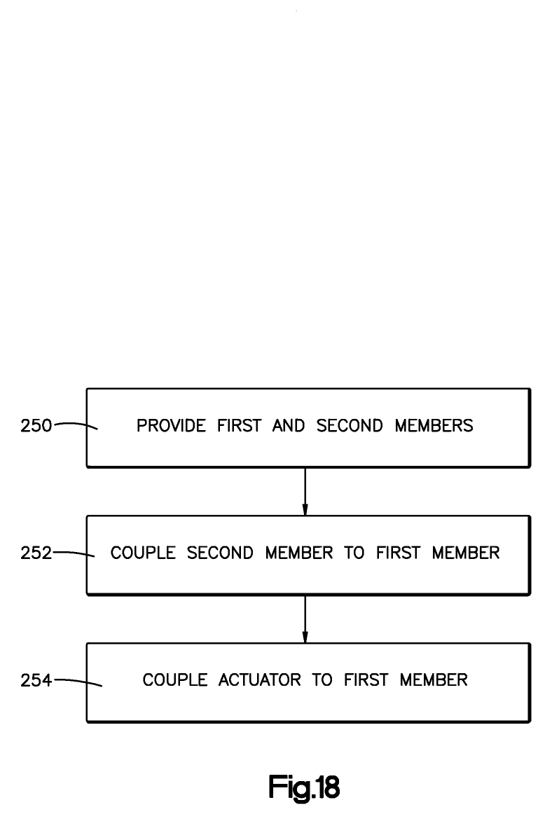

Referring to FIG. 18, a method of assembling the intramedullary nail 100 can include a step 250 of providing the nail body 118 and the pusher member 140. Providing the pusher member can include coupling the shaft 150 to the receiver 148. The method can include a step 252 of coupling the pusher member 140 to the nail body 118. The coupling step 252 can include positioning the pusher member 140 within the channel 138 of the nail body 118. The coupling step 252 can include positioning the second biasing element 168 in the channel 138 prior to positioning the pusher member 140 in the channel 138.

The method can include a step 254 of coupling the actuator 176 to the nail body 118. The coupling step 254 can include positioning the biasing element 182 within the channel 138. The coupling step can include positioning the alignment member 190 within the actuator slot 188. The coupling step 254 can include coupling the alignment member 190 to the nail body 118. The coupling step 254 can include positioning the biasing element 182 within the channel 138. The coupling step 254 can include coupling the connector 184 to the nail body 118. The coupling step 254 can include moving the connector 184 relative to the nail body 118. The coupling step 254 can include moving the connector 184 relative to the nail body 118 so as to increase the potential energy of the biasing element 182.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, reference to values stated in ranges includes each and every value within that range.

It is to be appreciated that certain features which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

What is claimed:

1. An intramedullary nail that extends along a central axis, the intramedullary nail comprising:
a nail body configured to be positioned in a medullary canal of a bone, wherein the nail body defines a slot and at least one first bone fixation hole, the at least one bone fixation hole configured to receive a respective at least one first bone fixation element so as to couple the first nail member to a first bone portion, and the slot configured to receive a bone anchor that is configured to be coupled to a second bone portion;
a biasing element configured to apply a biasing force to at least one of the bone anchor and the nail body, thereby creating compression between the first and second bone portions when the first nail member is coupled to the first bone portion and the bone anchor is received in the slot and coupled to the second bone portion.

2. The intramedullary nail of claim 1, wherein the biasing element applies the biasing force to the nail body.

3. The intramedullary nail of claim 2, wherein the biasing element applies the biasing force to the bone anchor.

4. The intramedullary nail of claim 1, further comprising an actuator coupled to the biasing element and the bone anchor such that the biasing element applies the biasing force to the actuator, thereby applying the biasing force to the bone anchor.

5. The intramedullary nail of claim 4, wherein the first nail member includes a channel configured to receive at least a portion of the actuator.

6. The intramedullary nail of claim 4, further comprising a pusher member configured to apply a second force to the bone anchor.

7. The intramedullary nail of claim 6, wherein the second force is applied in a first direction and the biasing force is applied in a second direction different from the first direction.

8. The intramedullary nail of claim 7, wherein the first direction is opposite the second direction.

9. The intramedullary nail of claim 6, wherein the second force is greater than the biasing force.

10. The intramedullary nail of claim 6, wherein the pusher member is configured to move the second bone portion away from the first bone portion.

11. The intramedullary nail of claim 6, wherein the pusher member is movable relative to the first nail member.

12. The intramedullary nail of claim 11, wherein the pusher member is moveable in a longitudinal direction relative to the first nail member.

13. The intramedullary nail of claim 6, wherein the biasing element and the pusher member are positioned on opposite sides of the bone anchor.

14. The intramedullary nail of claim 6, wherein the first nail member includes a bone fixation hole configured to receive a second bone anchor.

15. The intramedullary nail of claim 6, wherein the pusher member includes a pusher member slot configured to receive the second bone anchor.

16. The intramedullary nail of claim 1, wherein the biasing element is a first biasing element and the intramedullary nail further comprises a second biasing element.

17. The intramedullary nail of claim 16, wherein the first and second biasing elements are positioned on opposite sides of the slot in a longitudinal direction, and the second biasing element is positioned between the slot of the nail body and the first bone fixation hole in the longitudinal direction.

18. A method of applying compression between a first bone portion and a second bone portion, the method comprising:
inserting an intramedullary nail into a medullary canal of the bone;
inserting a bone anchor through a first slot in a nail body of the intramedullary nail into the first bone portion, such that the nail body is movable relative to the bone anchor along a central axis of the nail body;
inserting a first bone fixation element through the second bone portion into a first bone fixation hole in the nail body so as to couple the first nail member to the second bone portion; and
causing an actuator to bias against at least one of the bone anchor and the nail body the bone anchor so as to apply a compressive force between the bone anchor and the nail body.

19. The method of claim 18, wherein the first bone fixation hole is sized substantially equal to the first bone fixation element.

20. The method of claim 18, wherein the first bone fixation hole is elongate along the central axis.
